(19) United States
(12) Patent Application Publication
(10) Pub. No.: US 2024/0092987 A1
(43) Pub. Date: Mar. 21, 2024

(54) CONDUCTIVE ELASTOMERIC FOAM MATERIALS AND METHODS OF USE

(71) Applicant: Meta Platforms Technologies, LLC, Menlo Park, CA (US)

(72) Inventors: Li Yao, Bothell, WA (US); Wenyang Pan, Redmond, WA (US); Ziyan Liu, Renton, WA (US); Shawn Reese, Renton, WA (US); Thomas John Farrell Wallin, Redmond, WA (US); Zhenzhen Shen, Kirkland, WA (US); Fang He, Bothell, WA (US); Robert Cole Bolger-Cruz, Riverside, CA (US); Sudhanshu Rathod, Woodinville, WA (US); Kristopher Erickson, Sammamish, WA (US)

(21) Appl. No.: 18/467,201

(22) Filed: Sep. 14, 2023

Related U.S. Application Data
(60) Provisional application No. 63/375,766, filed on Sep. 15, 2022.

(51) Int. Cl. C08J 9/00; A61B 5/268

(52) U.S. Cl. CPC C08J 9/0076; A61B 5/268; C08K 3/046; C08K 2201/001; C08K 2201/011

(57) ABSTRACT

Described herein are conductive elastomeric foam materials and methods of making and using the same. The conductive elastomeric foam materials include a polymeric matrix, one or more conductive fillers, and one or more foaming agents. The polymeric matrix can include a thermoset polymer or a thermoplastic polymer. Also described herein are methods of making conductive elastomeric foam materials. Further described herein are molded products including the conductive elastomeric foam materials as described herein and wearable devices including the molded products.

### CROSS-REFERENCE TO PRIORITY APPLICATION

This application claims the benefit of and priority to U.S. Provisional Patent Application No. 63/375,766, filed Sep. 15, 2022, the contents of which are incorporated herein by reference in its entirety.

### BACKGROUND

Conductive elastomers are used for developing soft electrodes, soft actuators and soft sensors. Conductive elastomers are particularly important for electromyography (EMG) electrodes, which convert motoneuron signals into electrical signals. The electrical signals are then processed and amplified for external device control. Metal electrodes have been directly used for many applications; however, the metal electrodes are rigid and lack the necessary comfort for long-term (e.g., full day) wear. In addition, commercial conductive elastomers currently available fail to appropriately balance the required conductivity with the required compressibility. To maximize performance of EMG electrodes, high conductivity as well as good compatibility with the human body and skin are needed.

### SUMMARY

Described herein are conductive elastomeric foam materials and methods of making and using the same. A conductive elastomeric foam material as described herein comprises a polymeric matrix, one or more conductive fillers, and one or more foaming agents. The polymeric matrix optionally comprises a thermoset polymer or a thermoplastic polymer. The thermoset polymer can be selected from the group consisting of a silicone, a urethane, a urethane acrylate, an acrylate, a methacrylate, a polysulfide, a polythioether, an epoxy, a phenolic resin, and combinations thereof. Optionally, the thermoplastic polymer is selected from the group consisting of styrenic block copolymers, thermoplastic polyurethanes, polyethylenes, polypropylenes, polystyrenes, polyesters, polyamides, polyimides, polyphenylene sulfides, polyvinyl alcohols, polylactic acids, and thermoplastic silicones. In some cases, the thermoplastic polymer is a styrenic block copolymer and wherein the styrenic block copolymer is selected from the group consisting of a styrene-isoprene-styrene block polymer (SIS), a styrene-butadiene-styrene block polymer (SBS), a styrene-ethylene/butylene-styrene copolymer (SEBS), and a styrene-ethylene/proylene-styrene copolymer (SEPS).

The one or more conductive fillers for use in the conductive elastomeric foam materials described herein comprises a 1D filler, a 2D filler, or a combination thereof. The one or more conductive fillers optionally comprises an inorganic filler. In some cases, the inorganic filler comprises a carbon-based filler (e.g., carbon nanofibers, carbon nanotubes, carbon microfibers, carbon black, graphene, graphene oxide, or combinations thereof). Optionally, the one or more conductive fillers comprises silver flakes, silver nanowires, gold nanowires, poly(3,4-ethylenedioxythiophene) poly(styrenesulfonate) (PEDOT:PSS) nanofibrils, polyaniline nanofibrils, liquid metals, or combinations thereof.

The one or more foaming agents can comprise an inorganic foaming agent. The inorganic foaming agent can comprise an inorganic thermal decomposition blowing agent (e.g., bicarbonate, carbonate, nitrite, or a combination thereof) or an inorganic reactive blowing agent (e.g., sodium bicarbonate, zinc powder, hydrogen peroxide, or a yeast reaction). Optionally, the one or more foaming agents can comprise an organic foaming agent. The organic foaming agent can optionally comprise an azo foaming agent, a nitroso foaming agent, an acylhydrazide foaming agent, or a combination thereof. In some cases, the one or more foaming agents can comprise a microbead (e.g., expanded polylactide beads, expanded glass beads, polystyrene beads, unexpanded microbeads, or a combination thereof).

The conductive elastomeric foam materials can further comprise one or more additional additives. Optionally, the one or more additional additives can be selected from the group consisting of dispersants, plasticizers, polymer thinners, surfactants, thixotropic agents, and diluents. In some cases, the conductive elastomeric foam material comprises a plurality of pores. Optionally, an inner layer of the plurality of pores is coated with a coating material. The coating material can optionally be selected from the group consisting of gold, silver, titanium, poly(3,4-ethylenedioxythiophene) polystyrene sulfonate (PEDOT:PSS), polyaniline, polypyrrole, and a combination thereof.

Also described herein are methods of making a conductive elastomeric foam material as described herein. The methods of making a conductive elastomeric foam material comprise mixing a polymeric matrix, one or more conductive fillers, and one or more foaming agents to form a foamable conductive elastomeric material, and applying a foaming process to the foamable conductive elastomeric material to form a conductive elastomeric foam material having a plurality of pores. Optionally, the mixing is performed using speed-mixing, internal mixing, ball milling, planetary milling, roll-milling, or an attritor.

The foaming process can optionally comprise a mechanical foaming process, a pressure/heat foaming process, a supercritical fluid extraction foaming process, or a chemical foaming process. Optionally, the method of making the conductive elastomeric foam material can further comprise processing the conductive elastomeric foam material into a molded product. The processing can optionally be performed using compression molding, injection molding or dispensing, three-dimensional processing, freeform fabrication, or direct write extrusion. In some cases, the methods described herein can further comprise coating one or more surfaces of the foamable conductive elastomeric material with a coating material, the coating comprising dip-coating, ink-jet printing, slot-die coating, screen-printing, aerosol jetting, electrochemical deposition, or a surface treatment using oxygen plasma, a silane treatment, or a corona surface treatment.

Further described herein is a molded product, comprising a conductive elastomeric foam material as described herein. Optionally, the molded product comprises a cylindrical shape having a diameter from 1 mm to 10 mm (e.g., from 3 mm to 7 mm). In some cases, the molded product is a flat shape, a microneedle, a fuzzy structure, a dome shape, a hollow structure, or a cone shape. Optionally, at least one surface of the molded product is surface coated with a coating material. Optionally, a plurality of pores within the conductive elastomeric foam material is coated with a coating material. The coating material can be selected from the group consisting of poly(3,4-ethylenedioxythiophene) polystyrene sulfonate (PEDOT:PSS), a sulfonated tetrafluoroethylene based fluoropolymer-copolymer, a polymer binder, polyaniline, polypyrrole, silver nanowires (AgNW), gold nanowires (AuNW), liquid metal, and gold.

The molded product as described herein can exhibit a bulk conductivity of 10 Ohm-cm or lower. Optionally, the tensile strength of the molded product is 0.5 MPa or greater (e.g., 0.8 MPa or greater). In some cases, the modulus of the molded product is 5 MPa or lower (e.g., 2 MPa or lower). Optionally, the hardness of the molded product is 70 Shore A or lower (e.g., 50 Shore A or lower). The skin contact impedance of the molded product on the skin of a subject can be 1 MOhms or lower (e.g., 0.5 MOhms or lower) with a geometric contact area of 120 mm². Optionally, the skin contact impedance is no more than 20% higher than that of gold electrodes to skin. In some cases, the radial stiffness of the molded product is from 0.8 to 3 KPa (e.g., from 1 KPa to 2.25 KPa). The compression stroke of the molded product can be at least 50% (e.g., at least 80%). The stroke height of the molded product can be 0.5 mm or greater (e.g., 3.5 mm or greater). Optionally, the molded product exhibits a Z-conductivity of greater than 0.001 S/cm (e.g., greater than 0.01 S/cm or greater than 0.1 S/cm). In some cases, the molded product comprises an electrode (e.g., a biopotential electrode).

Further described herein is a wearable device comprising a molded product as described herein. Optionally, the wearable device can be a wristband or a monolithic conductive band. The wearable device can collect biopotential signals or electromyography signals.

The details of one or more embodiments are set forth in the drawings and the description below. Other features, objects, and advantages will be apparent from the description and drawings, and from the claims.

### BRIEF DESCRIPTION OF THE DRAWINGS

FIG. 1 is a depiction of the method of preparing the conductive foam material

FIG. 2 is a photograph of a conductive urethane acrylate foam loaded with 6 vol. % of carbon fiber

FIG. 3 contains photographs of a conductive foam elastomers formed into a flat cylindrical shape, both before and during compression with a weight.

### DETAILED DESCRIPTION

Described herein are conductive elastomeric foam materials and products prepared from the same. The conductive materials described herein are loaded with conductive fillers as well as foaming agents. The materials are processed in a manner to form uniform pores, which can optionally be coated, and can be further shaped into different designs (e.g., electrodes). In addition, electrochemical coating can be performed to coat further conductive layers on the inner layers of the pores in the foam elastomers, which further enhances the bulk conductivity and the contact impedance with skin. Surprisingly, the conductive foam elastomers and resulting materials and products exhibit a desirable balance of conductivity and compressibility, enabling long-term and comfortable wear for the user. The conductive foam elastomers described herein include a controlled and relatively low amount of filler as compared to other materials in the field, but still exhibit high conductivity and stretchability.

The conductive elastomeric foam materials described herein include a polymeric matrix, one or more conductive fillers, and one or more foaming agents. Each of the components of the materials, along with additional optional components, are further described below.

Polymeric Matrix

As noted above, the elastomeric foam materials described herein include a polymeric matrix. The polymeric matrix can be prepared from a thermoset polymer, a thermoplastic polymer, or a combination of a thermoset polymer and a thermoplastic polymer. As known to those of skill in the art, thermoplastic polymers are capable of melting and reflowing and are soluble in solvents. Thermoset polymers, after they are cured or crosslinked, are not soluble in solvents and will not reflow when heated. As further detailed herein, both types of polymers are suitable for use as the polymer matrix.

Thermoset polymers include polymer materials in which chemical reactions, including cross-linking, occur while the resins are being molded. The appearance, chemical properties, and physical properties of the final product are changed, and the product is generally resistant to further applications of heat. Optionally, the thermoset polymer for use in the elastomeric materials described herein can be a silicone, a urethane, a urethane acrylate, an acrylate, a methacrylate, a polysulfide, a polythioether, an epoxy, a phenolic resin, or any suitable combination of these.

In some cases, the polymeric matrix is or includes a thermoplastic polymer. Thermoplastic polymers are polymers that soften or become plastic when they are heated. The process of heating and cooling such polymers can be carried out repeatedly without affecting any appreciable change in the properties of the polymers. After thermoplastic polymers are synthesized, they can be dissolved in a solvent and applied to surfaces. Additionally, these polymers can be heated, causing them to melt flow and generally develop strong adhesive bonds to a substrate.

In some cases, the thermoplastic polymer is selected from the group consisting of styrenic block copolymers, thermoplastic polyurethanes, polyethylenes, polypropylenes, polystyrenes, polyesters, polyamides, polyimides, polyphenylene sulfides, polyvinyl alcohols, polylactic acids, and thermoplastic silicones. Optionally, the thermoplastic polymer can be styrenic block copolymers. By way of example, suitable styrenic block copolymers can be, for example, a styrene-isoprene-styrene block polymer (SIS), a styrene-butadiene-styrene block polymer (SBS), a styrene-ethylene/butylene-styrene copolymer (SEBS), and a styrene-ethylene/proylene-styrene copolymer (SEPS). Other thermoplastic polymers, as known in the art, can also be used as the polymeric matrix in the materials described herein.

Conductive Fillers

The conductive elastomeric materials described herein also include one or more conductive fillers, such as one dimensional (1D) fillers and/or two-dimensional (2D) fillers. In some cases, the conductive filler includes a 1D filler. Optionally, the conductive filler can include an inorganic filler. Optionally, the conductive filler can be a carbon-based filler, such as one or more of carbon nanofibers, carbon nanotubes, carbon microfibers, carbon black, graphene, graphene oxide, or combinations thereof. Optionally, the one or more confuctive fillers can be silver flakes, silver nanowires, gold nanowires, poly(3,4-ethylenedioxythiophene) poly(styrenesulfonate) (PEDOT:PSS) nanofibrils, polyaniline nanofibrils, liquid metals, or combinations thereof.

In some examples, one or more surfaces of the filler can be functionalized with a functional group, such as a hydroxyl group (—OH), a carboxylic group (—C(O)O—), a thiol group (—SH), or an amino group (—NH₂). The aforementioned functional groups can optionally be substituted with one or more groups. As used herein, the term substituted includes the addition of an alkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens, i.e., commensurate with its saturation level, with no substitutions, e.g., linear decane (—(CH₂)₉—CH₃).

The carbon-based fillers for use in the conductive elastomeric foam materials can have an appropriate size for the desired use. In some cases, the carbon nanofibers for use as the carbon-based fillers can have a diameter from 100 nm to 1000 nm (e.g., from 100 nm to 500 nm or from 130 nm to 200 nm). The diameter of the carbon nanofibers can be, for example 100 nm, 110 nm, 120 nm, 130 nm, 140 nm, 150 nm, 160 nm, 170 nm, 180 nm, 190 nm, 200 nm, 300 nm, 400 nm, 500 nm, 600 nm, 700 nm, 800 nm, 900 nm, or 1000 nm.

In some cases, the total amount of conductive filler present in the conductive elastomeric foam materials can be 50 wt. % or less (e.g., from 1 wt. % to 50 wt. %, from 2 wt. % to 35 wt. % or from 3 wt. % to 15 wt. %) based on the weight of the conductive elastomeric foam material. For example, the total amount of conductive filler present (e.g., the combined amount of all conductive filler types) can be 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 35 wt. %, 36 wt. %, 37 wt. %, 38 wt. %, 39 wt. %, 40 wt. %, 41 wt. %, 42 wt. %, 43 wt. %, 44 wt. %, 45 wt. %, 46 wt. %, 47 wt. %, 48 wt. %, 49 wt. %, or 50 wt. %.

Foaming Agents

The conductive elastomeric foam materials described herein also include one or more foaming agents. In some examples, the foaming agents include inorganic foaming agents. Suitable inorganic foaming agents include, for example, inorganic thermal decomposition blowing agents, such as bicarbonate, carbonate, nitrite, and the like. In other examples, suitable inorganic foaming agents include inorganic reactive blowing agents. The inorganic reactive blowing agents can be, for example, sodium bicarbonate, zinc powder, hydrogen peroxide, or a yeast reaction.

In some cases, the one or more foaming agents comprise an organic foaming agent. Suitable organic foaming agents include, for example, an azo foaming agent, a nitroso foaming agent, an acylhydrazide foaming agent, and the like. Optionally, the one or more foaming agents can include a microbead (e.g., an expandable microbead or a nonexpendable microbead). In some cases, the microbead can be or can include expanded polylactide beads, expanded glass beads, polystyrene beads, unexpanded microbeads, or a combination thereof.

The foaming agents can be present in the materials described herein in an amount of 10 wt. % or less based on the weight of the conductive elastomeric foam material. For example, one or more foaming agents can be included in an amount of 0.01 wt. % to 10 wt. %, 0.1 wt. % to 8 wt. %, 0.5 wt. % to 5 wt. %, or 1 wt. % to 3 wt. % based on the weight of the conductive elastomeric foam material.

Additional Additives

The conductive elastomeric foam materials described herein can optionally include one or more additional additives. Suitable additives for inclusion in the materials described herein can be, for example, one or more of dispersants, plasticizers, polymer thinners, surfactants, thixotropic agents, and diluents.

In some cases, the additional additive includes a dispersant. The dispersant can function to stabilize the inorganic filler particles in the composition. In some cases, without dispersant, components may aggregate and thus adversely affect the characteristics of the materials described herein. Suitable dispersants vary and depend on the specific identity and surface chemistry of conductive filler. In some cases, suitable dispersants include at least a binding group and a compatibilizing segment. The binding group can optionally be ionically bonded to the particle surface. Examples of binding groups include, for example, phosphoric acids, phosphonic acids, sulfonic acids, carboxylic acids, and amines. The compatibilizing segment can optionally be selected to be miscible with the polymeric matrix.

Additional additives for use in the materials describe herein can include hardeners, accelerators, thickeners, humectants, desiccants, fire retardants, electrical insulators, vibration dampeners, thermal insulators, corrosion inhibitors, antioxidants, pigments, dyes, magnetic particles, thermochromic agents (i.e., compounds that can change color with changing temperature), mechanochromic agents (i.e., compounds that can change color under mechanical deformation), anti-glare agents, anti-reflective agents, infrared reflective agents, stealth agents, textural agents, fragrances, self-cleaning agents, hydrophobic agents, hydrophilic agents, or any combination thereof.

The additional additives can be present in the materials described herein in an amount of 10 wt. % or less based on the weight of the conductive elastomeric foam material. For example, one or more additional additives can be included in an amount of 0.01 wt. % to 10 wt. %, 0.1 wt. % to 8 wt. %, 0.5 wt. % to 5 wt. %, or 1 wt. % to 3 wt. % based on the weight of the conductive elastomeric foam material. In some cases, for example for certain diluents and softeners (e.g., silicone oil and mineral oil), the content of the additive can be up to 30 wt. % (e.g., from 1 wt. % to 30 wt. %, from 5 wt. % to 25 wt. %, or from 10 wt. % to 20 wt. %).

Methods of Making and Resulting Products

Also described herein are methods of making the conductive elastomeric foam materials described above. The methods for making the conductive elastomeric foam materials as described herein can include a step of mixing a polymeric matrix, one or more conductive fillers, and one or more foaming agents, in the requisite amounts as detailed in the present disclosure. The polymeric matrix can be included in the mixture in an amount ranging from about 50% to about 95% based on the weight of the mixture. For example, the polymeric matrix can be present in the mixture in an amount of about 50%, about 55%, about 60%, about 65%, about 70%, about 75%, about 80%, about 85%, about 90%, or about 95%, based on the weight of the polymer mixture. The filler and optionally any suitable additive mentioned above can be included in their indicated amounts.

The mixing can be performed using any suitable apparatus for the selected components, along with the selected amounts (e.g., laboratory-scale or process-scale). In some examples, the mixing can be performed using speed-mixing, internal mixing, ball milling, planetary milling, roll-milling, or an attritor. The mixing is performed for a suitable period of time to result in a foamable conductive elastomeric material.

Following the mixing step, a foaming process can be applied to the foamable conductive elastomeric material to form a conductive elastomeric foam material. The foaming process can be performed by any suitable foaming technique as known to those in the art, including mechanical foaming, pressure/heat foaming, supercritical fluid extraction foaming, or chemical foaming. In a mechanical foaming process, air is added into the polymeric matrix by mechanical stirring. Physical foaming includes the application of pressure or heat, optionally with the addition of supercritical fluid addition. Chemical foaming includes the triggering or creation of a chemical reaction which generates air. Optionally, expandable and nonexpendable microbeads can be used to facilitate foaming. In some examples, a core-shell bead can be used, in which the core contains air. The shell can optionally be melted.

The resulting conductive elastomeric foam material has a plurality of pores. The average pore size of the pores in the foam material can range from 50 micron to 500 micron (e.g., from 100 micron to 400 micron or from 150 micron to 300 micron). Optionally, the average pore size is 50 micron, 60 micron, 70 micron, 80 micron, 90 micron, 100 micron, 110 micron, 120 micron, 130 micron, 140 micron, 150 micron, 160 micron, 170 micron, 180 micron, 190 micron, 200 micron, 210 micron, 220 micron, 230 micron, 240 micron, 250 micron, 260 micron, 270 micron, 280 micron, 290 micron, 300 micron, 310 micron, 320 micron, 330 micron, 340 micron, 350 micron, 360 micron, 370 micron, 380 micron, 390 micron, 400 micron, 410 micron, 420 micron, 430 micron, 440 micron, 450 micron, 460 micron, 470 micron, 480 micron, 490 micron, or 500 micron.

Optionally, a coating process can be performed before the foaming step or after the foaming step. In the coating process, an inner layer of the plurality of pores can be coated with a coating material. Optionally, the coating material is selected from the group consisting of gold, silver, titanium, poly(3,4-ethylenedioxythiophene) polystyrene sulfonate (PEDOT:PSS), polyaniline, polypyrrole, and a combination thereof.

After the mixing, foaming, and optional coating steps are performed, the resulting foamed material can be further processed into a molded product, such as a soft electrode. The processing can be performed using, for example, compression molding, injection molding or dispensing, three-dimensional processing, freeform fabrication, or direct write extrusion.

The molded product can have any suitable shape, and can be dictated by the end use of the product. In some cases, the molded product can have a cylindrical shape. Optionally, the cylindrical shape can have a diameter ranging from, for example, 1 mm to 10 mm (3 mm to 7 mm). Other suitable shapes include, for example, a flat shape, a microneedle, a fuzzy structure, a dome shape, a hollow structure, or a cone shape. Optionally, the molded product can be an electrode.

Optionally, the processing can further comprise coating one or more surfaces of the material with a polymer or another coating material. In some cases, at least one surface of the molded product is surface coated with a coating material. In other cases, a plurality of pores within the conductive elastomeric foam material can be coated with a coating material. The coating material can be, for example poly(3,4-ethylenedioxythiophene) polystyrene sulfonate (PEDOT:PSS), a sulfonated tetrafluoroethylene based fluoropolymer-copolymer, a polymer binder, polyaniline, polypyrrole, silver nanowires (AgNW), gold nanowires (AuNW), liquid metal, or gold. In some cases, the coating can be performed by using dip-coating, ink-jet printing, slot-die coating, screen-printing, aerosol jetting, electrochemical deposition, or a surface treatment using oxygen plasma, a silane treatment, a corona surface treatment, or any other suitable method.

Optionally, the molded product exhibits a bulk conductivity of 10 Ohm-cm or lower as determined by ASTM D991 (2020). For example, the molded product can have a bulk conductivity of 10 Ohm-cm or lower, 9 Ohm-cm or lower, 8 Ohm-cm or lower, 7 Ohm-cm or lower, 6 Ohm-cm or lower, 5 Ohm-cm or lower, 4 Ohm-cm or lower, 3 Ohm-cm or lower, 2 Ohm-cm or lower, 1 Ohm-cm or lower, or 0.5 Ohm-cm or lower. In some cases, the bulk conductivity is from 0.5 Ohm-cm to 10 Ohm-cm (e.g., from 1 Ohm-cm to 9 Ohm-cm or 2 Ohm-cm to 7 Ohm-cm).

The tensile strength of the molded products prepared from the conductive elastomeric materials described herein can be 0.5 MPa or greater (e.g., 0.8 MPa or greater) as determined by ASTM D624 (2020). For example, the tensile strength of the molded products can be 0.5 MPa, 0.55 MPa, 0.6 MPa, 0.65 MPa, 0.7 MPa, 0.75 MPa, 0.8 MPa, 0.85 MPa, 0.9 MPa, 0.95 MPa, or 1 MPa.

In some cases, the modulus of the molded product is 5 MPa or lower as determined by ASTM D624 (2020). For example, the molded product can have a modulus of 5 MPa or lower, 4 MPa or lower, 3 MPa or lower, 2 MPa or lower, or 1 MPa or lower. In some cases, the modulus is from 0.5 MPa to 5 MPa, 1 MPa to 4 MPa, or 2 MPa to 3 MPa.

The hardness of the molded product can be 70 Shore A or lower as determined by ASTM D2240-15 (2021). For example, the hardness of the molded product can be 70 Shore A or lower, 65 Shore A or lower, 60 Shore A or lower, 55 Shore A or lower, 50 Shore A or lower, 45 Shore A or lower, 40 Shore A or lower, 35 Shore A or lower, or 30 Shore A or lower.

The skin contact impedance of the molded product on the skin of a subject can be 1 MOhms or lower (e.g., 0.5 MOhms or lower) with a geometric contact area of 120 mm². Optionally, the skin contact impedance is comparable to that of gold electrodes to skin. For example, the skin contact impedance can be within 20% (e.g., 20% higher) than that of gold electrodes to skin), such as 15% higher, 10% higher, 5% higher, 4% higher, 3% higher, 2% higher, 1% higher, or exhibit the same skin contact impedance as gold electrodes.

Optionally, the radial stiffness of the molded product can be from 0.8 to 3 KPa. For example, the radial stiffness can be from 1 KPa to 3 KPa, from 1.5 KPa to 2.5 KPa, or from 1.7 KPa to 2.25 KPa. In some cases, the radial stiffness can be 0.8 KPa, 0.9 KPa, 1 KPa, 1.25 KPa, 1.5 KPa, 1.75 KPa, 2 KPa, 2.25 KPa, 2.5 KPa, 2.75 KPa, or 3 KPa.

The molded product can optionally display a compression stroke of at least 50%. For example, the compression stroke can be 55% or greater, 60% or greater, 65% or greater, 70% or greater, 75% or greater, 80% or greater, 85% or greater, 90% or greater, or 95% or greater. In some cases, the compression stroke range can be from 50% to 95%, from 55% to 90%, from 60% to 85%, from 65% to 95%, from 70% to 90%, from 75% to 85%, from 80% to 100%, from 80% to 95%, from 80% to 90%, from 85% to 100%, from 85% to 95%, or from 90% to 100%.

The stroke height of the molded product can be any suitable stroke height, such as 0.5 mm or greater (e.g., 1.0 mm or greater, 1.5 mm or greater, 2.0 mm or greater, 2.5 mm or greater, 3.0 mm or greater, or 3.5 mm or greater).

The molded product can optionally exhibit a Z-conductivity of greater than 0.001 S/cm (e.g., greater than 0.01 S/cm or greater than 0.1 S/cm) when measured at 142 Hz while being compressed by 12N of force between 2 parallel 304 stainless steel plates.

The molded products described herein can be integrated into a wearable device. Optionally, the wearable devices can be used to collect biopotential signals and/or electromyography signals. Suitable wearable devices include, for example, a wristband. Optionally, the molded product can be a monolithic conductive band, in which the elastomer is molded directly into the band rather than incorporating electrodes into the band. Specifically, electrodes are not needed in the monolithic conductive band since the entirety of the band is actively conductive, thus ensuring high surface area for maximal and effective contact. Beneficially, the monolithic conductive band also minimizes noise which may interfere with signal collection.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Fabricated Conductive Foam Elastomers

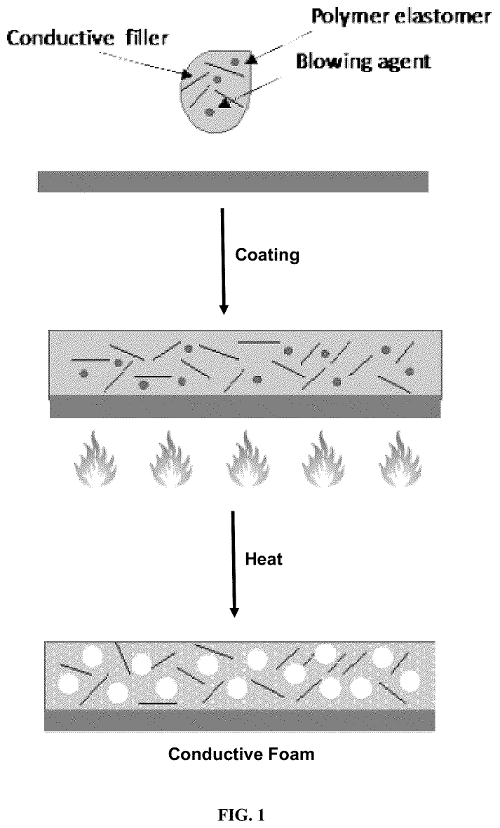

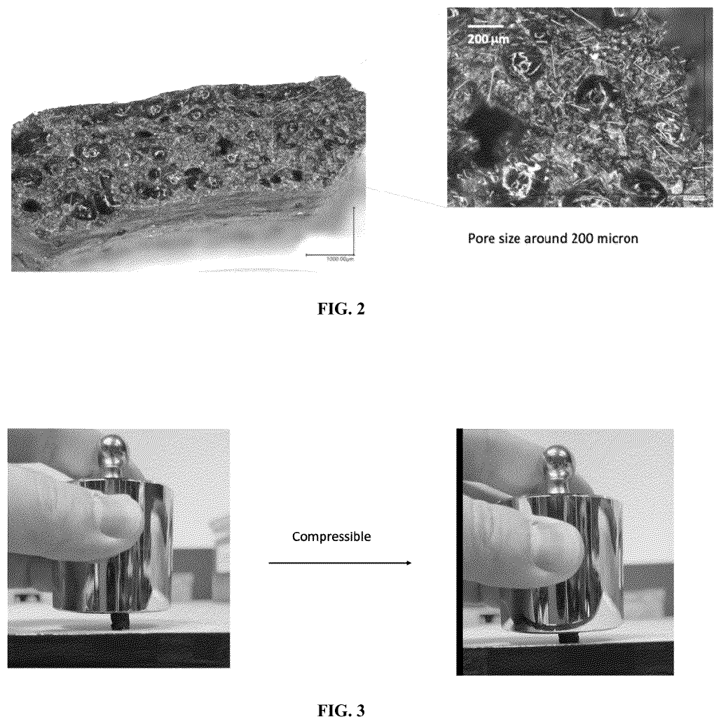

Conductive foam elastomers were fabricated by loading a urethane acrylate elastomer with 6 vol. % carbon nanofibers and an amount of a dual functional curing and blowing agent. The resulting material was then coated with a coating agent, and subjected to heating to result in a conductive foam material. See FIG. 1 for a depiction of the method of preparing the conductive foam material. The resulting conductive elastomeric foam material had a pore size of around 200 micron on average. See FIG. 2.

The mechanical properties of the resulting loaded elastomers were measured, including the tensile strength, elongation at break, Young's modulus, hardness, and resistivity. As shown in Table 1, elastomers can be successfully loaded with conductive fillers and subsequently foamed to achieve desired properties as dictated by the intended use for the elastomer.

TABLE 1

| | Sample 1 (Urethane Acrylate Elastomer |
|---|---|
| | and 6 vol. % Carbon Fiber) |
| Tensile Strength (MPa) | 0.85 |
| Elongation at Break (%) | 139 |
| Young's Modulus (MPa) | 1.9 |
| Hardness (Shore A) | 30 |
| Resistivity (Ohm/square) | 1.0E+04 |

### Example 2: Electrode Fabrication Method

Sample 1, fabricated as detailed above in Example 1, was formed into a soft biopotential electrode. Specifically, compression molding was used to prepare flat surface electrodes. The prepared flat surface electrodes were cylindrical in shape. As shown in FIG. 3, the flat cylindrical shape electrodes maintained compressibility.

### Exemplary Embodiments

1. A conductive elastomeric foam material, comprising:
   a polymeric matrix;
   one or more conductive fillers; and
   one or more foaming agents.
2. The conductive elastomeric foam material of embodiment 1, wherein the polymeric matrix comprises a thermoset polymer or a thermoplastic polymer.
3. The conductive elastomeric foam material of embodiment 2, wherein the polymeric matrix comprises a thermoset polymer.
4. The conductive elastomeric foam material of embodiment 3, wherein the thermoset polymer is selected from the group consisting of a silicone, a urethane, a urethane acrylate, an acrylate, a methacrylate, a polysulfide, a polythioether, an epoxy, a phenolic resin, and combinations thereof.
5. The conductive elastomeric foam material of embodiment 2, wherein the polymeric matrix comprises a thermoplastic polymer.
6. The conductive elastomeric foam material of embodiment 5, wherein the thermoplastic polymer is selected from the group consisting of styrenic block copolymers, thermoplastic polyurethanes, polyethylenes, polypropylenes, polystyrenes, polyesters, polyamides, polyimides, polyphenylene sulfides, polyvinyl alcohols, polylactic acids, and thermoplastic silicones.
7. The conductive elastomeric foam material of embodiment 6, wherein the thermoplastic polymer is a styrenic block copolymer and wherein the styrenic block copolymer is selected from the group consisting of a styrene-isoprene-styrene block polymer (SIS), a styrene-butadiene-styrene block polymer (SBS), a styrene-ethylene/butylene-styrene copolymer (SEBS), and a styrene-ethylene/proylene-styrene copolymer (SEPS).
8. The conductive elastomeric foam material of any one of embodiments 1-7, wherein the one or more conductive fillers comprises a 1D filler, a 2D filler, or a combination thereof.
9. The conductive elastomeric foam material of any one of embodiments 1-8, wherein the one or more conductive fillers comprises an inorganic filler.
10. The conductive elastomeric foam material of embodiment 9, wherein the inorganic filler comprises a carbon-based filler.
11. The conductive elastomeric foam material of embodiment 10, wherein the carbon-based filler comprises carbon nanofibers, carbon nanotubes, carbon microfibers, carbon black, graphene, graphene oxide, or combinations thereof.
12. The conductive elastomeric foam material of any one of embodiments 1-11, wherein the one or more conductive fillers comprises silver flakes, silver nanowires, gold nanowires, poly(3,4-ethylenedioxythiophene) poly(styrenesulfonate) (PEDOT:PSS) nanofibrils, polyaniline nanofibrils, liquid metals, or combinations thereof.
13. The conductive elastomeric foam material of any one of embodiments 1-12, wherein the one or more foaming agents comprises an inorganic foaming agent.
14. The conductive elastomeric foam material of embodiment 13, wherein the inorganic foaming agent comprises an inorganic thermal decomposition blowing agent.
15. The conductive elastomeric foam material of embodiment 14, wherein the inorganic thermal decomposition blowing agent comprises bicarbonate, carbonate, nitrite, or a combination thereof.
16. The conductive elastomeric foam material of embodiment 13, wherein the inorganic foaming agent comprises an inorganic reactive blowing agent.
17. The conductive elastomeric foam material of embodiment 16, wherein the inorganic reactive blowing agent comprises sodium bicarbonate, zinc powder, hydrogen peroxide, or a yeast reaction.
18. The conductive elastomeric foam material of any one of embodiments 1-12, wherein the one or more foaming agents comprises an organic foaming agent.
19. The conductive elastomeric foam material of embodiment 18, wherein the organic foaming agent comprises an azo foaming agent, a nitroso foaming agent, an acylhydrazide foaming agent, or a combination thereof.
20. The conductive elastomeric foam material of any one of embodiments 1-12, wherein the one or more foaming agents comprises a microbead.
21. The conductive elastomeric foam material of embodiment 20, wherein the microbead comprises expanded polylactide beads, expanded glass beads, polystyrene beads, unexpanded microbeads, or a combination thereof.
22. The conductive elastomeric foam material of any one of embodiments 1-21, further comprising one or more additional additives.
23. The conductive elastomeric foam material of embodiment 22, wherein the one or more additional additives is selected from the group consisting of dispersants, plasticizers, polymer thinners, surfactants, thixotropic agents, and diluents.
24. The conductive elastomeric foam material of any one of embodiments 1-23, wherein the conductive elastomeric foam material comprises a plurality of pores.
25. The conductive elastomeric foam material of embodiment 24, wherein an inner layer of the plurality of pores is coated with a coating material.
26. The conductive elastomeric foam material of embodiment 25, wherein the coating material is selected from the group consisting of gold, silver, titanium, poly(3,4-ethylenedioxythiophene) polystyrene sulfonate (PEDOT:PSS), polyaniline, polypyrrole, and a combination thereof.
27. A method of making a conductive elastomeric foam material according to any one of embodiments 1-26, comprising:
   mixing a polymeric matrix, one or more conductive fillers, and one or more foaming agents to form a foamable conductive elastomeric material; and
   applying a foaming process to the foamable conductive elastomeric material to form a conductive elastomeric foam material having a plurality of pores.
28. The method of embodiment 27, wherein the mixing is performed using speed-mixing, internal mixing, ball milling, planetary milling, roll-milling, or an attritor
29. The method of embodiment 27 or 28, wherein the foaming process comprises a mechanical foaming process, a pressure/heat foaming process, a supercritical fluid extraction foaming process, or a chemical foaming process.
30. The method of any one of embodiments 27-29, further comprising processing the conductive elastomeric foam material into a molded product.
31. The method of embodiment 30, wherein the processing can be performed using compression molding, injection molding or dispensing, three-dimensional processing, freeform fabrication, or direct write extrusion.
32. The method of any one of embodiments 27-31, further comprising coating one or more surfaces of the foamable conductive elastomeric material with a coating material, the coating comprising dip-coating, ink-jet printing, slot-die coating, screen-printing, aerosol jetting, electrochemical deposition, or a surface treatment using oxygen plasma, a silane treatment, or a corona surface treatment.
33. A molded product, comprising a conductive elastomeric foam material according to any one of embodiments 1-26.
34. The molded product of embodiment 33, wherein the molded product comprises a cylindrical shape having a diameter from 1 mm to 10 mm.
35. The molded product of embodiment 34, wherein the molded product comprises a cylindrical shape having a diameter from 3 mm to 7 mm.
36. The molded product of embodiment 33, wherein the molded product is a flat shape, a microneedle, a fuzzy structure, a dome shape, a hollow structure, or a cone shape.
37. The molded product of any one of embodiments 33-36, wherein at least one surface of the molded product is surface coated with a coating material or a plurality of pores within the conductive elastomeric foam material is coated with a coating material.
38. The molded product of embodiment 37, wherein the coating material is selected from the group consisting of poly(3,4-ethylenedioxythiophene) polystyrene sulfonate (PEDOT:PSS), a sulfonated tetrafluoroethylene based fluoropolymer-copolymer, a polymer binder, polyaniline, polypyrrole, silver nanowires (AgNW), gold nanowires (AuNW), liquid metal, and gold.
39. The molded product of any one of embodiments 33-38, wherein the molded product exhibits a bulk conductivity of 10 Ohm-cm or lower.
40. The molded product of any one of embodiments 33-39, wherein the tensile strength of the molded product is 0.5 MPa or greater.
41. The molded product of any one of embodiments 33-40, wherein the tensile strength of the molded product is 0.8 MPa or greater.
42. The molded product of any one of embodiments 33-41, wherein the modulus of the molded product is 5 MPa or lower.
43. The molded product of any one of embodiments 33-42, wherein the modulus of the molded product is 2 MPa or lower.
44. The molded product of any one of embodiments 33-43, wherein the hardness of the molded product is 70 Shore A or lower.
45. The molded product of any one of embodiments 33-44, wherein the hardness of the molded product is 50 Shore A or lower.
46. The molded product of any one of embodiments 33-45, wherein the skin contact impedance of the molded product on the skin of a subject is 1 MOhms or lower (e.g., 0.5 MOhms or lower) with a geometric contact area of 120 mm².
47. The molded product of any one of embodiments 33-46, wherein the skin contact impedance is no more than 20% higher than that of gold electrodes to skin.
48. The molded product of any one of embodiments 33-47, wherein the radial stiffness of the molded product is from 0.8 to 3 KPa.
49. The molded product of any one of embodiments 33-48, wherein the radial stiffness of the molded product is from 1 KPa to 2.25 KPa.
50. The molded product of any one of embodiments 33-49, wherein the compression stroke of the molded product is at least 50%.
51. The molded product of any one of embodiments 33-50, wherein the compression stroke of the molded product is at least 80%.
52. The molded product of any one of embodiments 33-51, wherein the stroke height of the molded product is 0.5 mm or greater.
53. The molded product of any one of embodiments 33-52, wherein the stroke height of the molded product is 3.5 mm or greater.
54. The molded product of any one of embodiments 33-53, wherein the molded product exhibits a Z-conductivity of greater than 0.001 S/cm.
55. The molded product of any one of embodiments 33-54, wherein the molded product exhibits a Z-conductivity of greater than 0.1 S/cm.
56. The molded product of any one of embodiments 33-55, wherein the molded product comprises an electrode.
57. A wearable device, comprising a molded product of any one of embodiments 33-56 integrated into the device.
58. The wearable device of embodiment 57, wherein the wearable device is a wristband.
59. The wearable device of embodiment 57, wherein the wearable decide is a monolithic conductive band.
60. The wearable device of any one of embodiments 57-59, wherein the wearable device collects biopotential signals.
61. The wearable device of any one of embodiments 57-59, wherein the wearable device collects electromyography signals.
The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions, methods, and aspects of these compositions and methods are specifically described, other compositions and methods are intended to fall within the scope of the appended claims. Thus, a combination of steps, elements, components, or constituents can be explicitly mentioned herein; however, all other combinations of steps, elements, components, and constituents are included, even though not explicitly stated.

What is claimed is:

1. A conductive elastomeric foam material, comprising:
a polymeric matrix;
one or more conductive fillers; and
one or more foaming agents.

2. The conductive elastomeric foam material of claim 1, wherein the polymeric matrix comprises a thermoset polymer.

3. The conductive elastomeric foam material of claim 2, wherein the thermoset polymer is selected from the group consisting of a silicone, a urethane, a urethane acrylate, an acrylate, a methacrylate, a polysulfide, a polythioether, an epoxy, a phenolic resin, and combinations thereof.

4. The conductive elastomeric foam material of claim 1, wherein the polymeric matrix comprises a thermoplastic polymer.

5. The conductive elastomeric foam material of claim 4, wherein the thermoplastic polymer is selected from the group consisting of styrenic block copolymers, thermoplastic polyurethanes, polyethylenes, polypropylenes, polystyrenes, polyesters, polyamides, polyimides, polyphenylene sulfides, polyvinyl alcohols, polylactic acids, and thermoplastic silicones.

6. The conductive elastomeric foam material of claim 1, wherein the one or more conductive fillers comprises a 1D filler, a 2D filler, or a combination thereof.

7. The conductive elastomeric foam material of claim 1, wherein the one or more foaming agents comprises an inorganic foaming agent, an organic foaming agent, or a microbead.

8. A method of making a conductive elastomeric foam material according to claim 1, comprising:
mixing a polymeric matrix, one or more conductive fillers, and one or more foaming agents to form a foamable conductive elastomeric material; and
applying a foaming process to the foamable conductive elastomeric material to form a conductive elastomeric foam material having a plurality of pores.

9. A molded product, comprising a conductive elastomeric foam material according to claim 1.

10. The molded product of claim 9, wherein the molded product comprises a cylindrical shape having a diameter from 1 mm to 10 mm.

11. The molded product of claim 9, wherein the molded product is a flat shape, a microneedle, a fuzzy structure, a dome shape, a hollow structure, or a cone shape.

12. The molded product of claim 9, wherein at least one surface of the molded product is surface coated with a coating material or a plurality of pores within the conductive elastomeric foam material is coated with a coating material.

13. The molded product of claim 12, wherein the coating material is selected from the group consisting of poly(3,4-ethylenedioxythiophene) polystyrene sulfonate (PEDOT:PSS), a sulfonated tetrafluoroethylene based fluoropolymer-copolymer, a polymer binder, polyaniline, polypyrrole, silver nanowires (AgNW), gold nanowires (AuNW), liquid metal, and gold.

14. The molded product of claim 9, wherein the molded product exhibits a bulk conductivity of 10 Ohm-cm or lower.

15. The molded product of claim 9, wherein the molded product exhibits a tensile strength of 0.5 MPa or greater.

16. The molded product of claim 9, wherein the molded product exhibits a modulus of 5 MPa or lower.

17. The molded product of claim 9, wherein the molded product exhibits a hardness of 70 Shore A or lower.

18. The molded product of claim 9, wherein the molded product exhibits a compression stroke of at least 50%.

19. A wearable device, comprising a molded product of claim 9 integrated into the wearable device.

20. The wearable device of claim 19, wherein the wearable device is a wristband or a monolithic conductive band.
